(19) United States
(12) Patent Application Publication
(10) Pub. No.: US 2026/0100282 A1
(43) Pub. Date: Apr. 9, 2026

(54) MACHINE LEARNING SYSTEMS AND RELATED ASPECTS FOR THE DETECTION OF DISEASE STATES

(71) Applicant: THE JOHNS HOPKINS UNIVERSITY, Baltimore, MD (US)

(72) Inventors: Therese L. CANARES, Baltimore, MD (US); Mathias UNBERATH, Baltimore, MD (US); Roger D. SOBERANIS MUKUL, Baltimore, MD (US)

(21) Appl. No.: 19/112,143

(22) PCT Filed: Sep. 14, 2023
(86) PCT No.: PCT/US2023/032720

§ 371 Date: Mar. 14, 2025

Related U.S. Application Data
(60) Provisional application No. 63/375,978, filed on Sep. 16, 2022.

(51) Int. Cl. G16H 50/30

(52) U.S. Cl. CPC G16H 50/30

(57) ABSTRACT

Examples may provide an electronic neural network (ENN) that has been trained on a set of training data that comprises sets of features extracted from oral cavity-related data obtained from reference subjects. The oral cavity-related data obtained from the reference subjects are each labeled with a positive or negative disease state ground truth classification for a given reference subject. Predictions for a positive or negative disease state classification for the given reference subject are made based on the oral cavity-related data obtained from the given reference subject, which predictions are compared to the ground truth classification for the given reference subject when the ENN is trained. The ENN outputs a prediction score for the disease state in a test subject that is indicated by a set of features extracted from oral cavity-related data obtained from the test subject when the set of features is passed through the ENN.

### CROSS-REFERENCE TO RELATED APPLICATIONS

This application is the national stage entry of International Patent Application No. PCT/US2023/032720, filed on Sep. 14, 2023, and published as WO 2024/059184 A1 on Mar. 21, 2024, which claims the benefit of U.S. Provisional Patent Application Ser. No. 63/375,978, filed on Sep. 16, 2022, which are hereby incorporated by reference in their entireties.

### FIELD

This disclosure relates generally to machine learning, e.g., in the context of medical applications, such as pathology.

### BACKGROUND

Telehealth involves the use of digital information and communication technologies to access health care services from locations that are remote from healthcare providers, such as from the patient's home. The communication technologies typically include mobile devices, such as smartphones and tablet computers. Currently, the provision of telehealth services is limited, as most diagnostic tests are unavailable in the home setting. In the case of streptococcus pharyngitis (i.e., strep throat or bacterial tonsillitis), for example, the disease state is generally diagnosed with a rapid strep test or throat swab in which a patient with a sore throat needs to be evaluated in person to receive the diagnostic test, thus defeating the underlying telehealth objective of providing health care services from remote locations.

Trained clinicians are unable to accurately identify streptococcus pharyngitis from clinical assessment or clinical prediction tools alone. Clinical assessment has 51% accuracy of diagnosing streptococcus pharyngitis.¹ The maximum score on the Centor or McIsaac scales (clinical prediction tools) gives a positive predictive value of streptococcus pharyngitis of 51-56%, and is linked to overprescribing of antibiotics^{2,3}. Therefore, a need exists to support clinicians with objective prediction of streptococcus pharyngitis that exceeds a clinician's assessment and today's prediction tools.

Accordingly, it is apparent that there is a need for additional methods of detecting disease states, such as streptococcus pharyngitis, including from locations that are remote from the patient.

Existing systems that use machine learning models and image analysis to identify disease states have limitations. While there are deep-learning methods that automate segmentation of various types of anatomy, automated image segmentation methods for throat images do not currently exist.

### SUMMARY

The present disclosure provides, in certain aspects, an artificial intelligence (AI) system capable of generating prediction scores for disease states in test subjects. In some aspects, for example, the present disclosure provides a computational framework for generating prediction scores for streptococcus pharyngitis in test subjects that uses electronic neural networks that have been trained with features extracted from oral cavity-related data obtained from reference subjects. In some embodiments, patients with strep throat can receive a diagnostic test at home, for example, by uploading a picture or video of their throat exam using a mobile device, and the analysis of the uploaded data is performed by the computer program products and related systems disclosed herein. These and other aspects will be apparent upon a complete review of the present disclosure, including the accompanying figures.

According to various embodiments, a computer-implemented method of generating a prediction score for a disease state in a test subject is presented. The method includes: passing a first set of features extracted from oral cavity-related data obtained from a test subject through an electronic neural network, wherein the electronic neural network has been trained on a first set of training data that comprises a plurality of sets of features extracted from oral cavity-related data obtained from reference subjects, wherein the oral cavity-related data obtained from the reference subjects are each labeled with a positive or negative disease state ground truth classification for a given reference subject, and wherein one or more predictions for a positive or negative disease state classification for the given reference subject are made based on the oral cavity-related data obtained from the given reference subject, which predictions are compared to the ground truth classification for the given reference subject when the electronic neural network is trained; and outputting from the electronic neural network the prediction score for the disease state in the test subject indicated by the first set of features extracted from the oral cavity-related data from the test subject.

According to various embodiments, a computer-implemented method of generating a prediction score for streptococcus pharyngitis in a test subject is presented. The method includes: passing a first set of features extracted from oral cavity-related data obtained from a test subject through an electronic neural network, wherein the electronic neural network has been trained on a first set of training data that comprises a plurality of sets of features extracted from oral cavity-related data obtained from reference subjects, wherein the oral cavity-related data obtained from the reference subjects are each labeled with a positive or negative streptococcus pharyngitis ground truth classification for a given reference subject, and wherein one or more predictions for a positive or negative streptococcus pharyngitis classification for the given reference subject are made based on the oral cavity-related data obtained from the given reference subject, which predictions are compared to the ground truth classification for the given reference subject when the electronic neural network is trained; and outputting from the electronic neural network a prediction score for streptococcus pharyngitis in the test subject indicated by the first set of features extracted from the oral cavity-related data from the test subject.

Various optional features of the above embodiments include the following. Generating a therapy recommendation for the test subject based upon the prediction score output from the electronic neural network. Administering a therapy to the test subject based upon the prediction score output from the electronic neural network. The oral cavity-related data comprises oral cavity images. The oral cavity-related data comprises image data, demographic data, symptom data, physical examination data, or a combination thereof. The demographic data comprises one or more of: subject age and subject sex. The symptom data comprises one or more subject symptoms selected from the group consisting of: fever, throat pain, pain with swallowing, ability to eat, drooling, difficulty with saliva, headache, cough, abdominal pain, nausea, vomiting, runny nose, nasal congestion, loss of taste, loss of smell, rash, exposure to an individual with streptococcus, and number of days with symptoms. The physical examination data comprises one or more physical examination observations for a subject selected from the group consisting of: fever, erythematous oropharynx, tonsillar enlargement, tonsillar exudate, palatal petechiae, enlarged lymph nodes, rash, and strawberry tongue. The disease state comprises a bacterial infection, a viral infection, or a peritonsillar abscess. The bacterial infection comprises a Streptococcus infection, a Gonorrhea infection, a Chlamydia infection, or a combination thereof. The viral infection comprises a respiratory syncytial virus (RSV) infection, an Epstein-Barr virus (EBV) infection, an adenovirus infection, a coronavirus infection, a human metapneumovirus (HMPV) infection, a human parainfluenza virus (HPIV) infection, a rhinovirus infection, an enterovirus infection, or a combination thereof. The coronavirus infection comprises a severe acute respiratory syndrome coronavirus-2 (SARS-CoV-2) infection.

Various additional optional features of the above embodiments include the following. The prediction score comprises a probability of a positive or negative streptococcus pharyngitis classification for the test subject. The oral cavity-related data comprises oral cavity images from the test and reference subjects, which oral cavity images comprise a region of interest selected from the group consisting of: a throat area, a tonsil area, a tongue area, a palate area, uvula area, posterior oropharynx area, lips area, cheek area, and neck area. Generating a three-dimensional (3D) model of the region of interest from the oral cavity images. Generating one or more rendered images from the 3D model. Standardizing the rendered images. Generating an estimated volume of the region of interest from the 3D model. The first set of training data comprises the rendered images and/or the estimated volume of the region of interest. The oral cavity images from the test and reference subjects are obtained from videos of the test and reference subjects. Obtaining the videos using a mobile device. The test subject obtains the videos. The healthcare provider obtains the videos. The features comprise numerical vectors. The first set of training data comprises oral cavity images and the electronic neural network has been further trained on a second set of training data that comprises a plurality of sets of features extracted from numerical vectors representing sets of parameterized demographic data, symptom data, and/or physical examination data from the reference subjects and wherein the computer-implemented method further comprises passing a second set of features extracted from a numerical vector representing a set of parameterized demographic data, symptom data, and/or physical examination data from the test subject through the electronic neural network. The numerical vectors representing the set of parameterized demographic data, symptom data, and/or physical examination data from the reference subjects and from the test subject each comprise at least a 15-dimensional vector. Mapping the first and second sets of features to a bidimensional vector that corresponds to the prediction score for streptococcus pharyngitis in the test subject. The electronic neural network uses one or more algorithms selected from the group consisting of: a random forest algorithm, a support vector machine algorithm, a decision tree algorithm, a linear classifier algorithm, a logistic regression, a linear regression algorithm, and a polynomial regression algorithm.

According to various embodiments, a system for generating a prediction score for a disease state in a test subject using an electronic neural network is presented. The system includes a processor; and a memory communicatively coupled to the processor, the memory storing instructions which, when executed on the processor, perform operations including: passing a first set of features extracted from oral cavity-related data obtained from a test subject through an electronic neural network, wherein the electronic neural network has been trained on a first set of training data that comprises a plurality of sets of features extracted from oral cavity-related data obtained from reference subjects, wherein the oral cavity-related data obtained from the reference subjects are each labeled with a positive or negative disease state ground truth classification for a given reference subject, and wherein one or more predictions for a positive or negative disease state classification for the given reference subject are made based on the oral cavity-related data obtained from the given reference subject, which predictions are compared to the ground truth classification for the given reference subject when the electronic neural network is trained; and outputting from the electronic neural network a prediction score for the disease state in the test subject indicated by the first set of features extracted from the oral cavity-related data from the test subject.

According to various embodiments, a system for generating a prediction score for streptococcus pharyngitis in a test subject using an electronic neural network is presented. The system includes a processor; and a memory communicatively coupled to the processor, the memory storing instructions which, when executed on the processor, perform operations including: passing a first set of features extracted from oral cavity-related data obtained from a test subject through the electronic neural network, wherein the electronic neural network has been trained on a first set of training data that comprises a plurality of sets of features extracted from oral cavity-related data obtained from reference subjects, wherein the oral cavity-related data obtained from the reference subjects are each labeled with a positive or negative streptococcus pharyngitis ground truth classification for a given reference subject, and wherein one or more predictions for a positive or negative streptococcus pharyngitis classification for the given reference subject are made based on the oral cavity-related data obtained from the given reference subject, which predictions are compared to the ground truth classification for the given reference subject when the electronic neural network is trained; and outputting from the electronic neural network a prediction score for streptococcus pharyngitis in the test subject indicated by the first set of features extracted from the oral cavity-related data obtained from the test subject.

Various optional features of the above embodiments include the following. The instructions which, when executed on the processor, further perform operations comprising: generating a therapy recommendation for the test subject based upon the prediction score output from the electronic neural network. The oral cavity-related data comprises oral cavity images. The oral cavity-related data comprises image data, demographic data, symptom data, physical examination data, or a combination thereof. The demographic data comprises one or more of: subject age and subject sex. The symptom data comprises one or more subject symptoms selected from the group consisting of: fever, throat pain, pain with swallowing, ability to eat, drooling, difficulty with saliva, headache, cough, abdominal pain, nausea, vomiting, runny nose, nasal congestion, loss of taste, loss of smell, rash, exposure to an individual with streptococcus, and number of days with symptoms. The physical examination data comprises one or more physical examination observations for a subject selected from the group consisting of: fever, erythematous oropharynx, tonsillar enlargement, tonsillar exudate, palatal petechiae, enlarged lymph nodes, rash, and strawberry tongue. The disease state comprises a bacterial infection, a viral infection, or a peritonsillar abscess. The bacterial infection comprises a Streptococcus infection, a Gonorrhea infection, a Chlamydia infection, or a combination thereof. The viral infection comprises a respiratory syncytial virus (RSV) infection, an Epstein-Barr virus (EBV) infection, an adenovirus infection, a coronavirus infection, a human metapneumovirus (HMPV) infection, a human parainfluenza virus (HPIV) infection, a rhinovirus infection, an enterovirus infection, or a combination thereof. The coronavirus infection comprises a severe acute respiratory syndrome coronavirus-2 (SARS-CoV-2) infection.

Various additional optional features of the above embodiments include the following. The prediction score comprises a probability of a positive or negative streptococcus pharyngitis classification for the test subject. The oral cavity-related data comprises oral cavity images from the test and reference subjects, which oral cavity images comprise a region of interest selected from the group consisting of: a throat area, a tonsil area, a tongue area, a palate area, uvula area, posterior oropharynx area, lips area, cheek area, and neck area. The instructions which, when executed on the processor, further perform operations comprising: generating a three-dimensional (3D) model of the region of interest from the oral cavity images. The instructions which, when executed on the processor, further perform operations comprising: generating one or more rendered images from the 3D model. The instructions which, when executed on the processor, further perform operations comprising: standardizing the rendered images. The instructions which, when executed on the processor, further perform operations comprising: generating an estimated volume of the region of interest from the 3D model. The first set of training data comprises the rendered images and/or the estimated volume of the region of interest. The oral cavity images from the test and reference subjects are obtained from videos of the test and reference subjects. The features comprise numerical vectors. The first set of training data comprises oral cavity images and the electronic neural network has been further trained on a second set of training data that comprises a plurality of sets of features extracted from numerical vectors representing sets of parameterized demographic data, symptom data, and/or physical examination data from the reference subjects and wherein the computer-implemented method further comprises passing a second set of features extracted from a numerical vector representing a set of parameterized demographic data, symptom data, and/or physical examination data from the test subject through the electronic neural network. The numerical vectors representing the set of parameterized demographic data, symptom data, and/or physical examination data from the reference subjects and from the test subject each comprise at least a 15-dimensional vector. The instructions which, when executed on the processor, further perform operations comprising: mapping the first and second sets of features to a bidimensional vector that corresponds to the prediction score for streptococcus pharyngitis in the test subject. The electronic neural network uses one or more algorithms selected from the group consisting of: a random forest algorithm, a support vector machine algorithm, a decision tree algorithm, a linear classifier algorithm, a logistic regression, a linear regression algorithm, and a polynomial regression algorithm.

According to various embodiments, a computer readable media is presented. The computer readable media comprises non-transitory computer executable instructions which, when executed by at least one electronic processor, perform at least: passing a first set of features extracted from oral cavity-related data obtained from a test subject through an electronic neural network, wherein the electronic neural network has been trained on a first set of training data that comprises a plurality of sets of features extracted from oral cavity-related data obtained from reference subjects, wherein the oral cavity-related data obtained from the reference subjects are each labeled with a positive or negative disease state ground truth classification for a given reference subject, and wherein one or more predictions for a positive or negative disease state classification for the given reference subject are made based on the oral cavity-related data obtained from the given reference subject, which predictions are compared to the ground truth classification for the given reference subject when the electronic neural network is trained; and outputting from the electronic neural network a prediction score for the disease state in the test subject indicated by the first set of features extracted from the oral cavity-related data from the test subject.

According to various embodiments, a computer readable media is presented. The computer readable media comprises non-transitory computer executable instructions which, when executed by at least one electronic processor, perform at least: passing a first set of features extracted from oral cavity-related data obtained from a test subject through the electronic neural network, wherein the electronic neural network has been trained on a first set of training data that comprises a plurality of sets of features extracted from oral cavity-related data obtained from reference subjects, wherein the oral cavity-related data obtained from the reference subjects are each labeled with a positive or negative streptococcus pharyngitis ground truth classification for a given reference subject, and wherein one or more predictions for a positive or negative streptococcus pharyngitis classification for the given reference subject are made based on the oral cavity-related data obtained from the given reference subject, which predictions are compared to the ground truth classification for the given reference subject when the electronic neural network is trained; and outputting from the electronic neural network a prediction score for streptococcus pharyngitis in the test subject indicated by the first set of features extracted from the oral cavity-related data obtained from the test subject.

### DRAWINGS

The above and/or other aspects and advantages will become more apparent and more readily appreciated from the following detailed description of examples, taken in conjunction with the accompanying drawings, in which:

FIG. 1 is a flow chart that schematically shows exemplary method steps of generating a prediction score for a disease state in a test subject according to some aspects disclosed herein; and

FIG. 2 is a schematic diagram of an exemplary system suitable for use with certain aspects disclosed herein.

FIG. 3 is a schematic diagram of an exemplary image-based classifier suitable for use with certain aspects disclosed herein.

FIG. 4 is a schematic diagram of an exemplary multi-modal classifier suitable for use with certain aspects disclosed herein.

FIG. 5 is a schematic diagram of an exemplary clinical decision support system suitable for use with certain aspects disclosed herein.

### DEFINITIONS

In order for the present disclosure to be more readily understood, certain terms are first defined below. Additional definitions for the following terms and other terms may be set forth throughout the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

It is also to be understood that the terminology used herein is for the purpose of describing particular embodiments only and is not intended to be limiting. Further, unless defined otherwise, all technical and scientific terms used herein have the same meaning as commonly understood by one of ordinary skill in the art to which this disclosure pertains. In describing and claiming the methods, systems, and computer readable media, the following terminology, and grammatical variants thereof, will be used in accordance with the definitions set forth below.

Classifier. As used herein, “classifier” generally refers to algorithm computer code that receives, as input, test data and produces, as output, a classification of the input data as belonging to one or another class.

Data set: As used herein, “data set” refers to a group or collection of information, values, or data points related to or associated with one or more objects, records, and/or variables. In some embodiments, a given data set is organized as, or included as part of, a matrix or tabular data structure. In some embodiments, a data set is encoded as a feature vector corresponding to a given object, record, and/or variable, such as a given test or reference subject. For example, a medical data set for a given subject can include one or more observed values of one or more variables associated with that subject.

Electronic neural network: As used herein, “electronic neural network” refers to a machine learning algorithm or model that includes layers of at least partially interconnected artificial neurons (e.g., perceptrons or nodes) organized as input and output layers with one or more intervening hidden layers that together form a network that is or can be trained to classify data, such as test subject medical data sets (e.g., medical images or the like).

Labeled: As used herein, “labeled” in the context of data sets or points refers to data that is classified as, or otherwise associated with, having or lacking a given characteristic or property.

Machine Learning Algorithm: As used herein, “machine learning algorithm” generally refers to an algorithm, executed by computer, that automates analytical model building, e.g., for clustering, classification or pattern recognition. Machine learning algorithms may be supervised or unsupervised. Learning algorithms include, for example, artificial neural networks (e.g., back propagation networks), discriminant analyses (e.g., Bayesian classifier or Fisher's analysis), multiple-instance learning (MIL), support vector machines, decision trees (e.g., recursive partitioning processes such as CART-classification and regression trees, or random forests), linear classifiers (e.g., multiple linear regression (MLR), partial least squares (PLS) regression, and principal components regression), hierarchical clustering, and cluster analysis. A dataset on which a machine learning algorithm learns can be referred to as “training data.” A model produced using a machine learning algorithm is generally referred to herein as a “machine learning model.”

Subject: As used herein, “subject” or “test subject” refers to an animal, such as a mammalian species (e.g., human) or avian (e.g., bird) species. More specifically, a subject can be a vertebrate, e.g., a mammal such as a mouse, a primate, a simian or a human. Animals include farm animals (e.g., production cattle, dairy cattle, poultry, horses, pigs, and the like), sport animals, and companion animals (e.g., pets or support animals). A subject can be a healthy individual, an individual that has or is suspected of having a disease or pathology or a predisposition to the disease or pathology, or an individual that is in need of therapy or suspected of needing therapy. The terms “individual” or “patient” are intended to be interchangeable with “subject.” A “reference subject” refers to a subject known to have or lack specific properties (e.g., a known pathology, such as melanoma and/or the like).

Value: As used herein, “value” generally refers to an entry in a dataset that can be anything that characterizes the feature to which the value refers. This includes, without limitation, numbers, words or phrases, symbols (e.g., + or −) or degrees.

### DESCRIPTION OF THE EMBODIMENTS

Reference will now be made in detail to example implementations. These embodiments are described in sufficient detail to enable those skilled in the art to practice the invention and it is to be understood that other embodiments may be utilized and that changes may be made without departing from the scope of the invention. The following description is, therefore, merely exemplary.

### I. Introduction

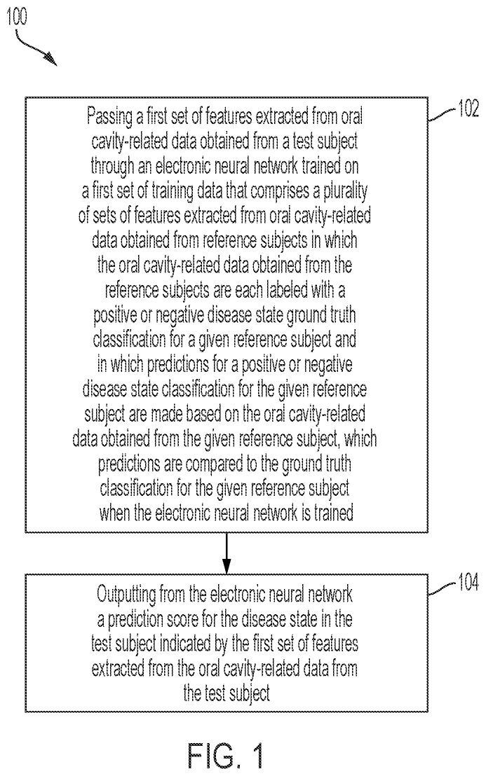

In some aspects, the present disclosure provides computer-implemented methods of generating a prediction score for a disease state in a test subject. To illustrate, FIG. 1 is a flow chart that schematically shows certain of these exemplary method steps. As shown, method 100 includes passing a first set of features extracted from oral cavity-related data obtained from a test subject through an electronic neural network that has been trained on a first set of training data that includes a plurality of sets of features extracted from oral cavity-related data obtained from reference subjects (step 102). Typically, the features extracted from the oral cavity-related data obtained from the test and reference subjects comprise numerical vectors. The oral cavity-related data obtained from the reference subjects are each labeled with a positive or negative disease state ground truth classification for a given reference subject. Also, one or more predictions for a positive or negative disease state classification for the given reference subject are made based on the oral cavity-related data obtained from the given reference subject, which predictions are compared to the ground truth classification for the given reference subject when the electronic neural network is trained. In some embodiments, the oral cavity-related data obtained from the test subject is received by a system that comprises the electronic neural network from a location that is remote from the system, such as from the test subject's home. In some embodiments, the electronic neural network uses one or more algorithms selected from, for example, a random forest algorithm, a support vector machine algorithm, a decision tree algorithm, a linear classifier algorithm, a logistic regression, a linear regression algorithm, a polynomial regression algorithm, or the like. Method 100 also includes outputting from the electronic neural network the prediction score for the disease state in the test subject indicated by the first set of features extracted from the oral cavity-related data from the test subject (step 104). In some embodiments, the disease state is streptococcus pharyngitis or strep throat. In some embodiments, method 100 also includes generating a therapy recommendation for the test subject based upon the prediction score output from the electronic neural network, such as when there is a positive indication of the presence of the disease state in the test subject. In some of these embodiments, method 100 further includes administering a therapy (e.g., an antibiotic therapy or the like) to the test subject based upon the prediction score output from the electronic neural network.

Various types of oral cavity-related data are optionally utilized in performing the methods of the present disclosure. In some embodiments, for example, the oral cavity-related data comprises oral cavity images. In some embodiments, the oral cavity-related data comprises image data, demographic data, symptom data, physical examination data, or a combination thereof. In some embodiments, the demographic data comprises one or more of: subject age and subjectsex. In some embodiments, the symptom data comprises one or more subject symptoms (i.e., symptoms exhibited by the subject, such as a test or reference subject), including, for example, fever, throat pain, pain with swallowing, ability to eat, drooling, difficulty with saliva, headache, cough, abdominal pain, nausea, vomiting, runny nose, nasal congestion, loss of taste, loss of smell, rash, exposure to an individual with streptococcus, and number of days with symptoms, among other symptom data. In some embodiments, the physical examination data comprises one or more physical examination observations for a subject (e.g., a test or reference subject) selected from, for example, fever, erythematous oropharynx, tonsillar enlargement, tonsillar exudate, palatal petechiae, enlarged lymph nodes, rash, strawberry tongue, and the like.

In some embodiments, images are preprocessed for training the classifiers (e.g., an image classifier model) disclosed herein. In some embodiments, the computation programs of the present disclosure automate identification of the frames of a video with the target region in focus and the frames are extracted for training the model. In some embodiments, the same procedure is to extract frames for analysis by the model (i.e., analyzing image data obtained from test subjects). In some embodiments, a user applies a bounding box to the target region of a given image, which may include, for example, a combination of tonsils, uvula, palate, tongue, lips, posterior oropharynx, neck, cheeks, or the like. In some embodiments, the algorithm applies automated segmentation in which the target region of interest is outlined and identified for analysis. Typically, each target region is labeled with the contents of that particular region.

In some embodiments, the bounding box of a target region is exported as coordinates in a text file. Since not all frames contain bounding boxes, the frames with bounding boxes and annotations must be extracted. A computer program reads the exported files for a set of images and verifies all its individual frames and associated text files. If the annotation text file contains bounding boxes, the program saves the frame in a different location. Additionally, it writes a new text file indicating the location of the video, the annotation type (tonsils, tongue, etc), and the bounding box coordinates. The program processes all the image sequences and organizes the information in a single file. These files are contained an index for sorting. Once they are retrieved, these files are used as a training set to develop an automated segmentation model.

The methods are related aspects of the present disclosure can be used to generate prediction scores for a wide range of disease states. In some embodiments, for example, the disease state comprises a bacterial infection, a viral infection, or a peritonsillar abscess. In some embodiments, the bacterial infection comprises a Streptococcus infection, a Gonorrhea infection, a Chlamydia infection, or a combination thereof. In some embodiments, the viral infection comprises a respiratory syncytial virus (RSV) infection, an Epstein-Barr virus (EBV) infection, an adenovirus infection, a coronavirus infection (e.g., a severe acute respiratory syndrome coronavirus-2 (SARS-CoV-2) infection, etc.), a human metapneumovirus (HMPV) infection, a human parainfluenza virus (HPIV) infection, a rhinovirus infection, an enterovirus infection, or a combination thereof.

In some embodiments, the prediction score comprises a probability of a positive or negative streptococcus pharyngitis classification or other disease state classification for the test subject. In some embodiments, the oral cavity-related data comprises oral cavity images from the test and reference subjects, which oral cavity images comprise a region of interest selected from the group consisting of: a throat area, a tonsil area, a tongue area, a palate area, uvula area, posterior oropharynx area, lips area, cheek area, and neck area. In some embodiments, the oral cavity images from the test and reference subjects are obtained from videos of the test and reference subjects. In some embodiments, the methods include obtaining the videos using a mobile device (e.g., a smartphone, a tablet computer, etc.). In some of these embodiments, the test subject obtains the videos (e.g., using their own mobile device), whereas in other embodiments, a healthcare provider or other third-party obtains the videos.

In some embodiments, the methods of the present disclosure include generating a three-dimensional (3D) model of the region of interest from the oral cavity images (e.g., using a neural radiance field (NeRF) technique or another approach). In some of these embodiments, the methods include generating one or more rendered images from the 3D model. In some embodiments, the methods include standardizing the rendered images. In some embodiments, the methods include generating an estimated volume of the region of interest (e.g., tonsil volume, etc.) from the 3D model. In some embodiments, the first set of training data used to train the electronic neural network comprises the rendered images and/or the estimated volume of the region of interest.

In some embodiments, the first set of training data comprises oral cavity images and the electronic neural network has been further trained on a second set of training data that comprises a plurality of sets of features extracted from numerical vectors representing sets of parameterized demographic data, symptom data, and/or physical examination data from the reference subjects and wherein the computer-implemented method further comprises passing a second set of features extracted from a numerical vector representing a set of parameterized demographic data, symptom data, and/or physical examination data from the test subject through the electronic neural network. In some embodiments, the numerical vectors representing the set of parameterized demographic data, symptom data, and/or physical examination data from the reference subjects and from the test subject each comprise at least a 15-dimensional vector (e.g., about a 20-dimensional vector, about a 25-dimensional vector, about a 30-dimensional vector, about a 35-dimensional vector, about a 40-dimensional vector, about a 45-dimensional vector, about a 50-dimensional vector, about a 60-dimensional vector, about a 70-dimensional vector, about an 80-dimensional vector, about a 90-dimensional vector, about a 100-dimensional vector, or more dimensional vector). In some embodiments, the methods of the present disclosure further include mapping the first and second sets of features to a bidimensional vector that corresponds to the prediction score for streptococcus pharyngitis in the test subject.

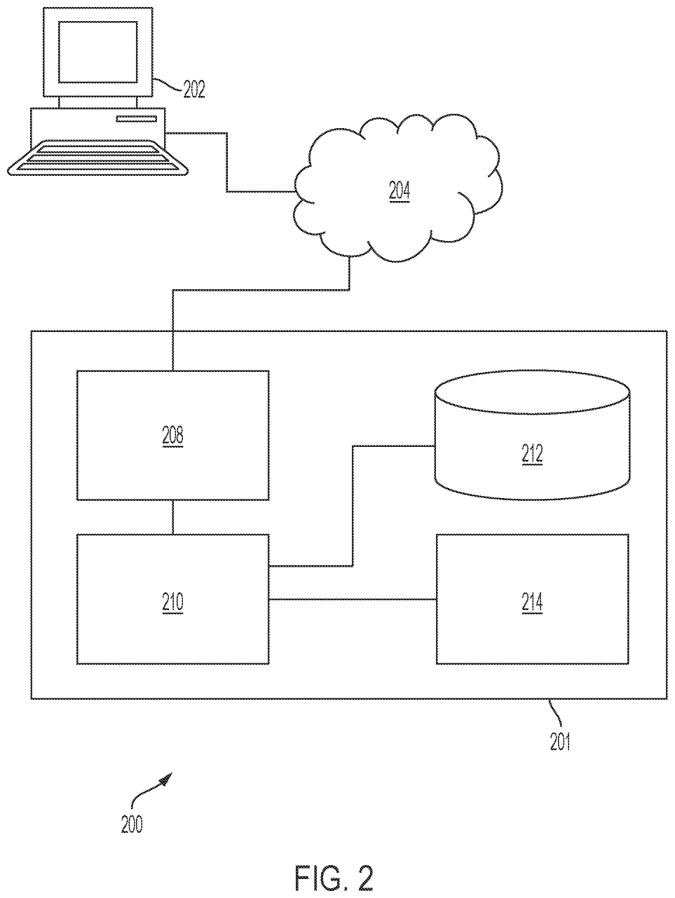

FIG. 2 is a schematic diagram of a hardware computer system 200 suitable for implementing various embodiments. For example, FIG. 2 illustrates various hardware, software, and other resources that can be used in implementations of any of methods disclosed herein, including method 100 and/or one or more instances of an electronic neural network. System 200 includes training corpus source 202 and computer 201. Training corpus source 202 and computer 201 may be communicatively coupled by way of one or more networks 204, e.g., the internet.

Training corpus source 202 may include an electronic clinical records system, such as an LIS, a database, a compendium of clinical data, or any other source of oral cavity-related data suitable for use as a training corpus as disclosed herein. According to some embodiments, each component is implemented as a vector, such as a feature vector, that represents a respective tile. Thus, the term “component” refers to both a tile and a feature vector representing a tile.

Computer 201 may be implemented as any of a desktop computer, a laptop computer, can be incorporated in one or more servers, clusters, or other computers or hardware resources, or can be implemented using cloud-based resources. Computer 201 includes volatile memory 214 and persistent memory 212, the latter of which can store computer-readable instructions, that, when executed by electronic processor 210, configure computer 201 to perform any of the methods disclosed herein, including method 100, and/or form or store any electronic neural network, and/or perform any classification technique as described herein. Computer 201 further includes network interface 208, which communicatively couples computer 201 to training corpus source 202 via network 204. Other configurations of system 200, associated network connections, and other hardware, software, and service resources are possible.

Certain embodiments can be performed using a computer program or set of programs. The computer programs can exist in a variety of forms both active and inactive. For example, the computer programs can exist as software program(s) comprised of program instructions in source code, object code, executable code or other formats; firmware program(s), or hardware description language (HDL) files. Any of the above can be embodied on a transitory or non-transitory computer readable medium, which include storage devices and signals, in compressed or uncompressed form. Exemplary computer readable storage devices include conventional computer system RAM (random access memory), ROM (read-only memory), EPROM (erasable, programmable ROM), EEPROM (electrically erasable, programmable ROM), and magnetic or optical disks or tapes.

### Image-Based Classifier

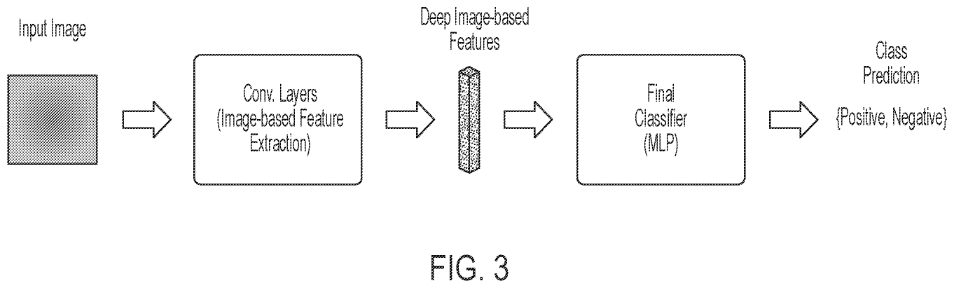

In some embodiments, the input images for training the models are obtained from a set of videos. The images are manually cropped into a region of interest containing only the throat area (potentially including tonsils and/or tongue). To further illustrate, FIG. 3 is a schematic diagram of an exemplary image-based classifier suitable for use with some of these embodiments.

In some embodiments, the model is composed of a deep convolutional neural network. It takes an image as input and outputs a two-dimensional vector representing the prediction scores for strep positive and negative, represented by the output of a SoftMax function. The architecture starts with a group of convolutional layers that learn to extract representative features from the input images. The extracted features are composed of high-dimensional numerical vectors that are then fed into a set of fully connected layers (MLP). The MLP learns a non-linear function that maps the input features into a two-dimensional vector indicating the prediction of the overall model (positive vs. negative). The overall model (convolutional and fully connected layers) follows a DenseNet architecture. Additionally, a ResNet architecture was also evaluated. Each artificial neuron (convolutional and fully connected MLP) of the image classifier is parametrized by a set of learnable weights.

In some embodiments, the weights are learned in an end-to-end way using the backpropagation algorithm. The algorithm employs a set of input images together with their corresponding actual class (positive or negative). During the training process, the model makes a prediction for the given input. This prediction is compared with the correct class (ground truth) to quantify the performance of the model. The quantification is done by employing an error or loss function. The learning process uses the derivatives of the loss function as a feedback signal for updating all the weights of the model in an iterative process. To account for class imbalance, a weighted cross-entropy loss function is employed.

### Multi-Modal Classifier

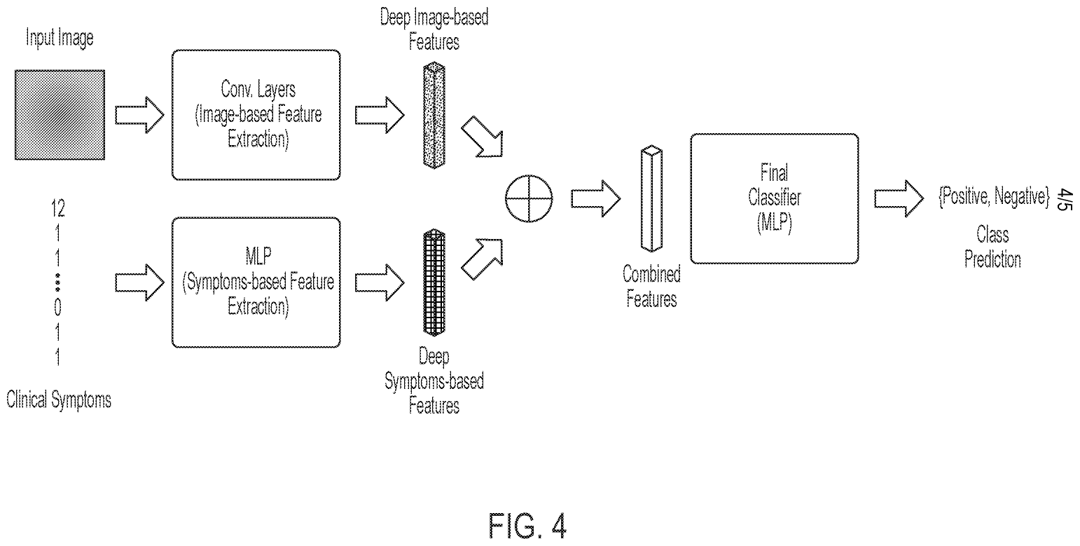

In some embodiments, the classifier employs two different inputs to perform the prediction (multi-modal approach). One input corresponds to a still frame following the same preprocessing described for the image-based classifier. The second input corresponds to a set of symptoms parametrized as, for example, a numerical 15-dimensional vector. To further illustrate, FIG. 4 is a schematic diagram of an exemplary multi-modal classifier suitable for use with some of these embodiments.

In some embodiments, the overall model comprises three sub-networks: two input branches and one for fusion and final classification. The input branches take the images and symptoms for feature extraction.

In some embodiments, the image-based feature extractor follows a similar architecture as the image classifier previously described, with the main difference that it does not include the fully connected layers. Hence, the image branch contains only convolutional layers. The architecture of this subnetwork is based on the ResNet or DenseNet networks (backbone model). The output of this branch is a feature vector representing the information extracted from the image. The size of this feature vector depends on the backbone employed.

In contrast, the symptoms-based feature extractor uses an MLP architecture is some embodiments. It takes a set of 15 clinical symptoms as input and outputs a feature representation encoded by a numerical vector. The size of the output feature vector is the same as the image-based feature vector.

In some embodiments, the final subnetwork is another MLP that takes the image-based and symptom-based feature vectors as input. This sub-network learns a function that maps the information from images and symptoms to a bidimensional vector, indicating the prediction score for positive and negative outcomes.

In some embodiments, all the sub-networks contain learnable weights that are optimized using the backpropagation algorithm.

### Clinical Decision Support System

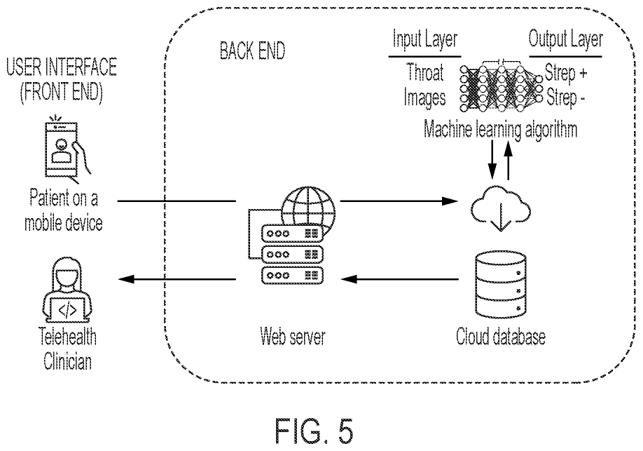

In some aspects, the present disclosure provides a clinical decision support system with telehealth applications. In some embodiments, a user uploads a oral cavity image (e.g., a throat image) recording to a web-based application. In some embodiments, that image is transmitted to a web server, and then transmitted to and stored on a cloud database. In some embodiments, key frames from the image and the target region of interest within those frames are isolated for analysis by the multi-modal classifier described herein. The classifier typically produces an output (class prediction) which is transmitted to the user on a device with an electronic display (mobile device or computer). In some embodiments, the back-end software is embedded into a telehealth software platform or may be a stand-alone web-based or mobile application. Users typically include test subjects (e.g., patients) or healthcare providers who may or may not be co-located. This system provides clinical decision support that combines the class prediction with guidance on medical management. Healthcare providers can use data from the clinical decision support system to inform clinical management. To further illustrate, FIG. 5 is a schematic diagram of an exemplary clinical decision support system suitable for use with some of these embodiments.

### II. Description of Example Embodiments

### Example: A Novel Method to Identify Streptococcus Pharyngitis Using Smartphone Images and Artificial Intelligence

### Background

Recent studies show artificial intelligence (AI) can identify streptococcal pharyngitis (strep throat) from a smartphone image, which has implications for telehealth. However, these studies lacked children (who more commonly experience strep throat), compared diseased vs. asymptomatic patients (instead of viral vs bacterial, which is more relevant in clinical practice), or used hardware devices, which limits generalizability to telehealth.

### Methods

This cross-sectional study was conducted at urban, tertiary care adult and pediatric emergency departments. In this convenience sample patients were included if a rapid antigen strep test or throat culture was ordered. The ground truth was defined as: positive strep throat if the rapid antigen or culture were positive; negative strep throat if the culture was negative. Centor/McIsaac scores and throat image(s) were recorded on a secure smartphone. If the strep throat test had not yet resulted, the clinician was asked their prediction of the strep throat test outcome.

The throat images were inputs used to train a deep convolutional neural network; the output was a binary prediction of positive/negative for strep throat. 1-3 images per patient comprised the dataset. The dataset was randomly divided for each phase of algorithm development: ˜50% (116 images) for training, ˜25% (59 images) for validation, and ˜25% (71 images) for testing. Results of the AI algorithm characteristics are reported on the testing image dataset.

Outcomes were sensitivity, specificity, positive predictive value (PPV), and negative predictive value (NPV) of the AI algorithm, Centor score, McIsaac score, and clinician prediction.

### Results

In total, 193 patients were approached, 4 declined consent/assent, and 5 had missing test data; 185 patients were included in the analysis. Patient, clinical score, and clinician characteristics are noted in Table 1. 83 clinician predictions were recorded; for the remainder, the strep throat test resulted before the provider could offer a prediction. The overall prevalence of strep throat was 25.4%.

The AI algorithm has a specificity similar to a Centor score of ≥3 (80% vs 85.5%, respectively), and exceeds the sensitivity, PPV and NPV of both Centor≥3 and clinician predictions (Table 2).

### CONCLUSIONS

This study demonstrates feasibility of an AI algorithm to predict strep throat in pediatric patients using a smartphone image. The current image-based AI algorithm is comparable to Centor scores and clinician predictions for sensitivity, specificity, PPV and NPV, which has implications as a clinical decision support tool. Future studies will focus on strategies to enhance AI performance such as an increased sample size or incorporating Centor criteria into the predictive model.

TABLE 1

| | | |
|---|---|---|
| Total | Negative Strep * | Positive Strep * |

Patient Characteristics
N = 185

| | |
|---|---|
| Age in years- Mean (Std Dev) | 11.4 (7.0) |

Sex N(%)

| | | | |
|---|---|---|---|
| Male | 83 (44.9%) | 64 (34.6%) | 19 (10.3%) |
| Female | 102 (55.1%) | 74 (40.0%) | 28 (15.1%) |

Race/Ethnicity (Patients may
select more than 1)

| | | | |
|---|---|---|---|
| African American | 138 (74.6%) | 101 (54.6%) | 37 (20.0%) |
| Caucasian | 27 (14.6%) | 22 (11.9%) | 5 (2.7%) |
| Latinx | 26 (14.1%) | 21 (11.4%) | 5 (2.7%) |
| Asian | 5 (2.7%) | 2 (1.6%) | 2 (1.1) |
| Native American/Alaskan | 0 (0%) | 0 (0%) | 0 (0%) |

Clinical Score Characteristics
Centor Score

| | | | |
|---|---|---|---|
| 0 | 27 (14.6%) | 23 (12.4%) | 4 (2.2%) |
| 1 | 69 (37.3%) | 56 (30.3%) | 13 (7.0%) |
| 2 | 54 (29.2%) | 39 (21.1) | 15 (8.1%) |
| 3 | 32 (17.3%) | 18 (9.7%) | 14 (7.6%) |
| 4 | 3 (1.6%) | 2 (1.1%) | 1 (0.5%) |

Mclsaac Score

| | | | |
|---|---|---|---|
| −1 | 0 (0%) | 0 (0%) | 0 (0%) |
| 0 | 6 (3.2%) | 6 (3.2%) | 0 (0%) |
| 1 | 41 (22.2%) | 34 (18.4%) | 7 (3.8%) |
| 2 | 64 (34.6%) | 47 (25.4%) | 17 (9.2%) |
| 3 | 51 (27.6%) | 35 (18.9%) | 16 (8.6%) |
| 4 | 22 (11.9%) | 15 (8.1%) | 7 (3.8%) |
| 5 | 1 (0.5%) | 1 (0.5%) | 0 (0%) |

Clinician Prediction
Characteristics N = 83
Sex N(%)

| | |
|---|---|
| Male | 18 (21.7%) |
| Female | 65 (78.3%) |

Degree

| | |
|---|---|
| MD/DO | 28 (33.7%) |
| PA | 55 (66.2%) |
| NP | 0 (0%) |

Years Since Degree

| | |
|---|---|
| PGY-1 | 13 (15.7%) |
| PGY-2 | 7 (8.4%) |
| PGY-3 | 25 (30.1%) |
| PGY-4+ or PA/NP 4+ years | 32 (38.6%) |
| Fellow/Attending | 6 (7.2%) |

* A negative strep throat was defined as negative throat culture; A positive strep throat was defined as positive antigen test or culture.
MD/DP, physician;
PA, physician assistant;
NP, nurse practitioner

TABLE 2

| | | | Positive | Negative |
|---|---|---|---|---|
| Test | | | Predictive | Predictive |
| Characteristics | Sensitivity | Specificity | Value | Value |
| AI Algorithm | 56.3% | 80.0% | 45.0% | 86.2% |

Centor Score

| | | | | |
|---|---|---|---|---|
| ≥1 | 91.5% | 16.7% | 27.2% | 85.2% |
| ≥2 | 63.8% | 57.2% | 33.7% | 82.3% |
| ≥3 | 31.9% | 85.5% | 42.9% | 78.7% |
| 4 | 2.1% | 98.6% | 33.3% | 74.7% |

Mclsaac Score

| | | | | |
|---|---|---|---|---|
| 0 | 100.0% | 0.0% | 25.4% | — |
| ≥1 | 100.0% | 4.3% | 26.3% | 100.0% |
| ≥2 | 85.1% | 29.0% | 29.0% | 85.1% |
| ≥3 | 48.9% | 63.0% | 31.1% | 78.4% |
| ≥4 | 14.9% | 88.4% | 30.4% | 75.3% |
| 5 | 0.0% | 99.3% | 0.0% | 74.5% |
| Clinician Prediction | 55.0% | 66.7% | 34.4% | 82.4% |

Some further aspects are defined in the following clauses:

Clause 1: A computer-implemented method of generating a prediction score for a disease state in a test subject, the method comprising: passing a first set of features extracted from oral cavity-related data obtained from a test subject through an electronic neural network, wherein the electronic neural network has been trained on a first set of training data that comprises a plurality of sets of features extracted from oral cavity-related data obtained from reference subjects, wherein the oral cavity-related data obtained from the reference subjects are each labeled with a positive or negative disease state ground truth classification for a given reference subject, and wherein one or more predictions for a positive or negative disease state classification for the given reference subject are made based on the oral cavity-related data obtained from the given reference subject, which predictions are compared to the ground truth classification for the given reference subject when the electronic neural network is trained; and, outputting from the electronic neural network the prediction score for the disease state in the test subject indicated by the first set of features extracted from the oral cavity-related data from the test subject.

Clause 2: The computer-implemented method of Clause 1, comprising generating a therapy recommendation for the test subject based upon the prediction score output from the electronic neural network.

Clause 3: The computer-implemented method of Clause 1 or Clause 2, comprising administering a therapy to the test subject based upon the prediction score output from the electronic neural network.

Clause 4: The computer-implemented method of any one of the preceding Clauses 1-3, wherein the oral cavity-related data comprises oral cavity images.

Clause 5: The computer-implemented method of any one of the preceding Clauses 1-4, wherein the oral cavity-related data comprises image data, demographic data, symptom data, physical examination data, or a combination thereof.

Clause 6: The computer-implemented method of any one of the preceding Clauses 1-5, wherein the demographic data comprises one or more of: subject age and subject sex.

Clause 7: The computer-implemented method of any one of the preceding Clauses 1-6, wherein the symptom data comprises one or more subject symptoms selected from the group consisting of: fever, throat pain, pain with swallowing, ability to eat, drooling, difficulty with saliva, headache, cough, abdominal pain, nausea, vomiting, runny nose, nasal congestion, loss of taste, loss of smell, rash, exposure to an individual with streptococcus, and number of days with symptoms.

Clause 8: The computer-implemented method of any one of the preceding Clauses 1-7, wherein the physical examination data comprises one or more physical examination observations for a subject selected from the group consisting of: fever, erythematous oropharynx, tonsillar enlargement, tonsillar exudate, palatal petechiae, enlarged lymph nodes, rash, and strawberry tongue.

Clause 9: The computer-implemented method of any one of the preceding Clauses 1-8, wherein the disease state comprises a bacterial infection, a viral infection, or a peritonsillar abscess.

Clause 10: The computer-implemented method of any one of the preceding Clauses 1-9, wherein the bacterial infection comprises a Streptococcus infection, a Gonorrhea infection, a Chlamydia infection, or a combination thereof.

Clause 11: The computer-implemented method of any one of the preceding Clauses 1-10, wherein the viral infection comprises a respiratory syncytial virus (RSV) infection, an Epstein-Barr virus (EBV) infection, an adenovirus infection, a coronavirus infection, a human metapneumovirus (HMPV) infection, a human parainfluenza virus (HPIV) infection, a rhinovirus infection, an enterovirus infection, or a combination thereof.

Clause 12: The computer-implemented method of any one of the preceding Clauses 1-11, wherein the coronavirus infection comprises a severe acute respiratory syndrome coronavirus-2 (SARS-CoV-2) infection.

Clause 13: The computer-implemented method of any one of the preceding Clauses 1-12, wherein the prediction score comprises a probability of a positive or negative streptococcus pharyngitis classification for the test subject.

Clause 14: The computer-implemented method of any one of the preceding Clauses 1-13, wherein the oral cavity-related data comprises oral cavity images from the test and reference subjects, which oral cavity images comprise a region of interest selected from the group consisting of: a throat area, a tonsil area, a tongue area, a palate area, uvula area, posterior oropharynx area, lips area, cheek area, and neck area.

Clause 15: The computer-implemented method of any one of the preceding Clauses 1-14, comprising generating a three-dimensional (3D) model of the region of interest from the oral cavity images.

Clause 16: The computer-implemented method of any one of the preceding Clauses 1-15, comprising generating one or more rendered images from the 3D model.

Clause 17: The computer-implemented method of any one of the preceding Clauses 1-16, comprising standardizing the rendered images.

Clause 18: The computer-implemented method of any one of the preceding Clauses 1-17, comprising generating an estimated volume of the region of interest from the 3D model.

Clause 19: The computer-implemented method of any one of the preceding Clauses 1-18, wherein the first set of training data comprises the rendered images.

Clause 20: The computer-implemented method of any one of the preceding Clauses 1-19, wherein the first set of training data comprises the estimated volume of the region of interest.

Clause 21: The computer-implemented method of any one of the preceding Clauses 1-20, wherein the oral cavity images from the test and reference subjects are obtained from videos of the test and reference subjects.

Clause 22: The computer-implemented method of any one of the preceding Clauses 1-21, comprising obtaining the videos using a mobile device.

Clause 23: The computer-implemented method of any one of the preceding Clauses 1-22, wherein the test subject obtains the videos.

Clause 24: The computer-implemented method of any one of the preceding Clauses 1-23, wherein a healthcare provider obtains the videos.

Clause 25: The computer-implemented method of any one of the preceding Clauses 1-24, wherein the features comprise numerical vectors.

Clause 26: The computer-implemented method of any one of the preceding Clauses 1-25, wherein the first set of training data comprises oral cavity images and wherein the electronic neural network has been further trained on a second set of training data that comprises a plurality of sets of features extracted from numerical vectors representing sets of parameterized demographic data, symptom data, and/or physical examination data from the reference subjects and wherein the computer-implemented method further comprises passing a second set of features extracted from a numerical vector representing a set of parameterized demographic data, symptom data, and/or physical examination data from the test subject through the electronic neural network.

Clause 27: The computer-implemented method of any one of the preceding Clauses 1-26, wherein the numerical vectors representing the set of parameterized demographic data, symptom data, and/or physical examination data from the reference subjects and from the test subject each comprise at least a 15-dimensional vector.

Clause 28: The computer-implemented method of any one of the preceding Clauses 1-27, further comprising mapping the first and second sets of features to a bidimensional vector that corresponds to the prediction score for the disease state in the test subject.

Clause 29: The computer-implemented method of any one of the preceding Clauses 1-28, wherein the electronic neural network uses one or more algorithms selected from the group consisting of: a random forest algorithm, a support vector machine algorithm, a decision tree algorithm, a linear classifier algorithm, a logistic regression, a linear regression algorithm, and a polynomial regression algorithm.

Clause 30: A computer-implemented method of generating a prediction score for streptococcus pharyngitis in a test subject, the method comprising: passing a first set of features extracted from oral cavity-related data obtained from a test subject through an electronic neural network, wherein the electronic neural network has been trained on a first set of training data that comprises a plurality of sets of features extracted from oral cavity-related data obtained from reference subjects, wherein the oral cavity-related data obtained from the reference subjects are each labeled with a positive or negative streptococcus pharyngitis ground truth classification for a given reference subject, and wherein one or more predictions for a positive or negative streptococcus pharyngitis classification for the given reference subject are made based on the oral cavity-related data obtained from the given reference subject, which predictions are compared to the ground truth classification for the given reference subject when the electronic neural network is trained; and, outputting from the electronic neural network a prediction score for streptococcus pharyngitis in the test subject indicated by the first set of features extracted from the oral cavity-related data from the test subject.

Clause 31: The computer-implemented method of Clause 30, comprising generating a therapy recommendation for the test subject based upon the prediction score output from the electronic neural network.

Clause 32: The computer-implemented method of Clause 30 or Clause 31, comprising administering a therapy to the test subject based upon the prediction score output from the electronic neural network.

Clause 33: The computer-implemented method of any one of the preceding Clauses 30-32, wherein the oral cavity-related data comprises oral cavity images.

Clause 34: The computer-implemented method of any one of the preceding Clauses 30-33, wherein the oral cavity-related data comprises image data, demographic data, symptom data, physical examination data, or a combination thereof.

Clause 35: The computer-implemented method of any one of the preceding Clauses 30-34, wherein the demographic data comprises one or more of: subject age and subject sex.

Clause 36: The computer-implemented method of any one of the preceding Clauses 30-35, wherein the symptom data comprises one or more subject symptoms selected from the group consisting of: fever, throat pain, pain with swallowing, ability to eat, drooling, difficulty with saliva, headache, cough, abdominal pain, nausea, vomiting, runny nose, nasal congestion, loss of taste, loss of smell, rash, exposure to an individual with streptococcus, and number of days with symptoms.

Clause 37: The computer-implemented method of any one of the preceding Clauses 30-36, wherein the physical examination data comprises one or more physical examination observations for a subject selected from the group consisting of: fever, erythematous oropharynx, tonsillar enlargement, tonsillar exudate, palatal petechiae, enlarged lymph nodes, rash, and strawberry tongue.

Clause 38: The computer-implemented method of any one of the preceding Clauses 30-37, wherein the prediction score comprises a probability of a positive or negative streptococcus pharyngitis classification for the test subject.

Clause 39: The computer-implemented method of any one of the preceding Clauses 30-38, wherein the oral cavity-related data comprises oral cavity images from the test and reference subjects, which oral cavity images comprise a region of interest selected from the group consisting of: a throat area, a tonsil area, a tongue area, a palate area, uvula area, posterior oropharynx area, lips area, cheek area, and neck area.

Clause 40: The computer-implemented method of any one of the preceding Clauses 30-39, comprising generating a three-dimensional (3D) model of the region of interest from the oral cavity images.

Clause 41: The computer-implemented method of any one of the preceding Clauses 30-40, comprising generating one or more rendered images from the 3D model.

Clause 42: The computer-implemented method of any one of the preceding Clauses 30-41, comprising standardizing the rendered images.

Clause 43: The computer-implemented method of any one of the preceding Clauses 30-42, comprising generating an estimated volume of the region of interest from the 3D model.

Clause 44: The computer-implemented method of any one of the preceding Clauses 30-43, wherein the first set of training data comprises the rendered images.

Clause 45: The computer-implemented method of any one of the preceding Clauses 30-44, wherein the first set of training data comprises the estimated volume of the region of interest.

Clause 46: The computer-implemented method of any one of the preceding Clauses 30-45, wherein the oral cavity images from the test and reference subjects are obtained from videos of the test and reference subjects.

Clause 47: The computer-implemented method of any one of the preceding Clauses 30-46, comprising obtaining the videos using a mobile device.

Clause 48: The computer-implemented method of any one of the preceding Clauses 30-47, wherein the test subject obtains the videos.

Clause 49: The computer-implemented method of any one of the preceding Clauses 30-48, wherein a healthcare provider obtains the videos.

Clause 50: The computer-implemented method of any one of the preceding Clauses 30-49, wherein the features comprise numerical vectors.

Clause 51: The computer-implemented method of any one of the preceding Clauses 30-50, wherein the first set of training data comprises oral cavity images and wherein the electronic neural network has been further trained on a second set of training data that comprises a plurality of sets of features extracted from numerical vectors representing sets of parameterized demographic data, symptom data, and/or physical examination data from the reference subjects and wherein the computer-implemented method further comprises passing a second set of features extracted from a numerical vector representing a set of parameterized demographic data, symptom data, and/or physical examination data from the test subject through the electronic neural network.

Clause 52: The computer-implemented method of any one of the preceding Clauses 30-51, wherein the numerical vectors representing the set of parameterized demographic data, symptom data, and/or physical examination data from the reference subjects and from the test subject each comprise at least a 15-dimensional vector.

Clause 53: The computer-implemented method of any one of the preceding Clauses 30-52, further comprising mapping the first and second sets of features to a bidimensional vector that corresponds to the prediction score for streptococcus pharyngitis in the test subject.

Clause 54: The computer-implemented method of any one of the preceding Clauses 30-53, wherein the electronic neural network uses one or more algorithms selected from the group consisting of: a random forest algorithm, a support vector machine algorithm, a decision tree algorithm, a linear classifier algorithm, a logistic regression, a linear regression algorithm, and a polynomial regression algorithm.

Clause 55: A system for generating a prediction score for a disease state in a test subject using an electronic neural network, the system comprising: a processor; and a memory communicatively coupled to the processor, the memory storing instructions which, when executed on the processor, perform operations comprising: passing a first set of features extracted from oral cavity-related data obtained from a test subject through an electronic neural network, wherein the electronic neural network has been trained on a first set of training data that comprises a plurality of sets of features extracted from oral cavity-related data obtained from reference subjects, wherein the oral cavity-related data obtained from the reference subjects are each labeled with a positive or negative disease state ground truth classification for a given reference subject, and wherein one or more predictions for a positive or negative disease state classification for the given reference subject are made based on the oral cavity-related data obtained from the given reference subject, which predictions are compared to the ground truth classification for the given reference subject when the electronic neural network is trained; and outputting from the electronic neural network a prediction score for the disease state in the test subject indicated by the first set of features extracted from the oral cavity-related data from the test subject.

Clause 56: The system of Clause 55, wherein the instructions which, when executed on the processor, further perform operations comprising: generating a therapy recommendation for the test subject based upon the prediction score output from the electronic neural network.

Clause 57: The system of Clause 55 or Clause 56, wherein the oral cavity-related data comprises oral cavity images.

Clause 58: The system of any one of the preceding Clauses 55-57, wherein the oral cavity-related data comprises image data, demographic data, symptom data, physical examination data, or a combination thereof.

Clause 59: The system of any one of the preceding Clauses 55-58, wherein the demographic data comprises one or more of: subject age and subject sex.

Clause 60: The system of any one of the preceding Clauses 55-59, wherein the symptom data comprises one or more subject symptoms selected from the group consisting of: fever, throat pain, pain with swallowing, ability to eat, drooling, difficulty with saliva, headache, cough, abdominal pain, nausea, vomiting, runny nose, nasal congestion, loss of taste, loss of smell, rash, exposure to an individual with streptococcus, and number of days with symptoms.

Clause 61: The system of any one of the preceding Clauses 55-60, wherein the physical examination data comprises one or more physical examination observations for a subject selected from the group consisting of: fever, erythematous oropharynx, tonsillar enlargement, tonsillar exudate, palatal petechiae, enlarged lymph nodes, rash, and strawberry tongue.

Clause 62: The system of any one of the preceding Clauses 55-61, wherein the disease state comprises a bacterial infection, a viral infection, or a peritonsillar abscess.

Clause 63: The system of any one of the preceding Clauses 55-62, wherein the bacterial infection comprises a Streptococcus infection, a Gonorrhea infection, a Chlamydia infection, or a combination thereof.

Clause 64: The system of any one of the preceding Clauses 55-63, wherein the viral infection comprises a respiratory syncytial virus (RSV) infection, an Epstein-Barr virus (EBV) infection, an adenovirus infection, a coronavirus infection, a human metapneumovirus (HMPV) infection, a human parainfluenza virus (HPIV) infection, a rhinovirus infection, an enterovirus infection, or a combination thereof.

Clause 65: The system of any one of the preceding Clauses 55-64, wherein the coronavirus infection comprises a severe acute respiratory syndrome coronavirus-2 (SARS-CoV-2) infection.

Clause 66: The system of any one of the preceding Clauses 55-65, wherein the prediction score comprises a probability of a positive or negative streptococcus pharyngitis classification for the test subject.

Clause 67: The system of any one of the preceding Clauses 55-66, wherein the oral cavity-related data comprises oral cavity images from the test and reference subjects, which oral cavity images comprise a region of interest selected from the group consisting of: a throat area, a tonsil area, a tongue area, a palate area, uvula area.

Clause 68: The system of any one of the preceding Clauses 55-67, wherein the instructions which, when executed on the processor, further perform operations comprising: generating a three-dimensional (3D) model of the region of interest from the oral cavity images.

Clause 69: The system of any one of the preceding Clauses 55-68, wherein the instructions which, when executed on the processor, further perform operations comprising: generating one or more rendered images from the 3D model.

Clause 70: The system of any one of the preceding Clauses 55-69, wherein the instructions which, when executed on the processor, further perform operations comprising: standardizing the rendered images.

Clause 71: The system of any one of the preceding Clauses 55-70, wherein the instructions which, when executed on the processor, further perform operations comprising: generating an estimated volume of the region of interest from the 3D model.

Clause 72: The system of any one of the preceding Clauses 55-71, wherein the first set of training data comprises the rendered images.

Clause 73: The system of any one of the preceding Clauses 55-72, wherein the first set of training data comprises the estimated volume of the region of interest.

Clause 74: The system of any one of the preceding Clauses 55-73, wherein the oral cavity images from the test and reference subjects are obtained from videos of the test and reference subjects.

Clause 75: The system of any one of the preceding Clauses 55-74, wherein the features comprise numerical vectors.

Clause 76: The system of any one of the preceding Clauses 55-75, wherein the first set of training data comprises oral cavity images and wherein the electronic neural network has been further trained on a second set of training data that comprises a plurality of sets of features extracted from numerical vectors representing sets of parameterized demographic data, symptom data, and/or physical examination data from the reference subjects and wherein the computer-implemented method further comprises passing a second set of features extracted from a numerical vector representing a set of parameterized demographic data, symptom data, and/or physical examination data from the test subject through the electronic neural network.

Clause 77: The system of any one of the preceding Clauses 55-76, wherein the numerical vectors representing the set of parameterized demographic data, symptom data, and/or physical examination data from the reference subjects and from the test subject each comprise at least a 15-dimensional vector.

Clause 78: The system of any one of the preceding Clauses 55-77, wherein the instructions which, when executed on the processor, further perform operations comprising: mapping the first and second sets of features to a bidimensional vector that corresponds to the prediction score for the disease state in the test subject.

Clause 79: The system of any one of the preceding Clauses 55-78, wherein the electronic neural network uses one or more algorithms selected from the group consisting of: a random forest algorithm, a support vector machine algorithm, a decision tree algorithm, a linear classifier algorithm, a logistic regression, a linear regression algorithm, and a polynomial regression algorithm.

Clause 80: A system for generating a prediction score for streptococcus pharyngitis in a test subject using an electronic neural network, the system comprising: a processor; and a memory communicatively coupled to the processor, the memory storing instructions which, when executed on the processor, perform operations comprising: passing a first set of features extracted from oral cavity-related data obtained from a test subject through the electronic neural network, wherein the electronic neural network has been trained on a first set of training data that comprises a plurality of sets of features extracted from oral cavity-related data obtained from reference subjects, wherein the oral cavity-related data obtained from the reference subjects are each labeled with a positive or negative streptococcus pharyngitis ground truth classification for a given reference subject, and wherein one or more predictions for a positive or negative streptococcus pharyngitis classification for the given reference subject are made based on the oral cavity-related data obtained from the given reference subject, which predictions are compared to the ground truth classification for the given reference subject when the electronic neural network is trained; and outputting from the electronic neural network a prediction score for streptococcus pharyngitis in the test subject indicated by the first set of features extracted from the oral cavity-related data obtained from the test subject.

Clause 81: The system of Clause 80, wherein the instructions which, when executed on the processor, further perform operations comprising: generating a therapy recommendation for the test subject based upon the prediction score output from the electronic neural network.

Clause 82: The system of Clause 80 or Clause 81, wherein the oral cavity-related data comprises oral cavity images.

Clause 83: The system of any one of the preceding Clauses 80-82, wherein the oral cavity-related data comprises image data, demographic data, symptom data, physical examination data, or a combination thereof.

Clause 84: The system of any one of the preceding Clauses 80-83, wherein the demographic data comprises one or more of: subject age and subject sex.

Clause 85: The system of any one of the preceding Clauses 80-84, wherein the symptom data comprises one or more subject symptoms selected from the group consisting of: fever, throat pain, pain with swallowing, ability to eat, drooling, difficulty with saliva, headache, cough, abdominal pain, nausea, vomiting, runny nose, nasal congestion, loss of taste, loss of smell, rash, exposure to an individual with streptococcus, and number of days with symptoms.

Clause 86: The system of any one of the preceding Clauses 80-85, wherein the physical examination data comprises one or more physical examination observations for a subject selected from the group consisting of: fever, erythematous oropharynx, tonsillar enlargement, tonsillar exudate, palatal petechiae, enlarged lymph nodes, rash, and strawberry tongue.

Clause 87: The system of any one of the preceding Clauses 80-86, wherein the prediction score comprises a probability of a positive or negative streptococcus pharyngitis classification for the test subject.

Clause 88: The system of any one of the preceding Clauses 80-87, wherein the oral cavity-related data comprises oral cavity images from the test and reference subjects, which oral cavity images comprise a region of interest selected from the group consisting of: a throat area, a tonsil area, a tongue area, a palate area, uvula area.

Clause 89: The system of any one of the preceding Clauses 80-88, wherein the instructions which, when executed on the processor, further perform operations comprising: generating a three-dimensional (3D) model of the region of interest from the oral cavity images.

Clause 90: The system of any one of the preceding Clauses 80-89, wherein the instructions which, when executed on the processor, further perform operations comprising: generating one or more rendered images from the 3D model.

Clause 91: The system of any one of the preceding Clauses 80-90, wherein the instructions which, when executed on the processor, further perform operations comprising: standardizing the rendered images.

Clause 92: The system of any one of the preceding Clauses 80-91, wherein the instructions which, when executed on the processor, further perform operations comprising: generating an estimated volume of the region of interest from the 3D model.

Clause 93: The system of any one of the preceding Clauses 80-92, wherein the first set of training data comprises the rendered images.

Clause 94: The system of any one of the preceding Clauses 80-93, wherein the first set of training data comprises the estimated volume of the region of interest.

Clause 95: The system of any one of the preceding Clauses 80-94, wherein the oral cavity images from the test and reference subjects are obtained from videos of the test and reference subjects.

Clause 96: The system of any one of the preceding Clauses 80-95, wherein the features comprise numerical vectors.

Clause 97: The system of any one of the preceding Clauses 80-96, wherein the first set of training data comprises oral cavity images and wherein the electronic neural network has been further trained on a second set of training data that comprises a plurality of sets of features extracted from numerical vectors representing sets of parameterized demographic data, symptom data, and/or physical examination data from the reference subjects and wherein the computer-implemented method further comprises passing a second set of features extracted from a numerical vector representing a set of parameterized demographic data, symptom data, and/or physical examination data from the test subject through the electronic neural network.

Clause 98: The system of any one of the preceding Clauses 80-97, wherein the numerical vectors representing the set of parameterized demographic data, symptom data, and/or physical examination data from the reference subjects and from the test subject each comprise at least a 15-dimensional vector.

Clause 99: The system of any one of the preceding Clauses 80-98, wherein the instructions which, when executed on the processor, further perform operations comprising: mapping the first and second sets of features to a bidimensional vector that corresponds to the prediction score for streptococcus pharyngitis in the test subject.

Clause 100: The system of any one of the preceding Clauses 80-99, wherein the electronic neural network uses one or more algorithms selected from the group consisting of: a random forest algorithm, a support vector machine algorithm, a decision tree algorithm, a linear classifier algorithm, a logistic regression, a linear regression algorithm, and a polynomial regression algorithm.

Clause 101: A computer readable media comprising non-transitory computer executable instructions which, when executed by at least one electronic processor, perform at least: passing a first set of features extracted from oral cavity-related data obtained from a test subject through an electronic neural network, wherein the electronic neural network has been trained on a first set of training data that comprises a plurality of sets of features extracted from oral cavity-related data obtained from reference subjects, wherein the oral cavity-related data obtained from the reference subjects are each labeled with a positive or negative disease state ground truth classification for a given reference subject, and wherein one or more predictions for a positive or negative disease state classification for the given reference subject are made based on the oral cavity-related data obtained from the given reference subject, which predictions are compared to the ground truth classification for the given reference subject when the electronic neural network is trained; and outputting from the electronic neural network a prediction score for the disease state in the test subject indicated by the first set of features extracted from the oral cavity-related data from the test subject.

Clause 102: A computer readable media comprising non-transitory computer executable instructions which, when executed by at least one electronic processor, perform at least: passing a first set of features extracted from oral cavity-related data obtained from a test subject through the electronic neural network, wherein the electronic neural network has been trained on a first set of training data that comprises a plurality of sets of features extracted from oral cavity-related data obtained from reference subjects, wherein the oral cavity-related data obtained from the reference subjects are each labeled with a positive or negative streptococcus pharyngitis ground truth classification for a given reference subject, and wherein one or more predictions for a positive or negative streptococcus pharyngitis classification for the given reference subject are made based on the oral cavity-related data obtained from the given reference subject, which predictions are compared to the ground truth classification for the given reference subject when the electronic neural network is trained; and outputting from the electronic neural network a prediction score for streptococcus pharyngitis in the test subject indicated by the first set of features extracted from the oral cavity-related data obtained from the test subject.

While the invention has been described with reference to the exemplary embodiments thereof, those skilled in the art will be able to make various modifications to the described embodiments without departing from the true spirit and scope. The terms and descriptions used herein are set forth by way of illustration only and are not meant as limitations. In particular, although the method has been described by examples, the steps of the method can be performed in a different order than illustrated or simultaneously. Those skilled in the art will recognize that these and other variations are possible within the spirit and scope as defined in the following claims and their equivalents.

1. A computer-implemented method of generating a prediction score for a disease state in a test subject, the method comprising:
passing a first set of features extracted from oral cavity-related data obtained from a test subject through an electronic neural network, wherein the electronic neural network has been trained on a first set of training data that comprises a plurality of sets of features extracted from oral cavity-related data obtained from reference subjects, wherein the oral cavity-related data obtained from the reference subjects are each labeled with a positive or negative disease state ground truth classification for a given reference subject, and wherein one or more predictions for a positive or negative disease state classification for the given reference subject are made based on the oral cavity-related data obtained from the given reference subject, which predictions are compared to the ground truth classification for the given reference subject when the electronic neural network is trained; and,
outputting from the electronic neural network the prediction score for the disease state in the test subject indicated by the first set of features extracted from the oral cavity-related data from the test subject.

2. The computer-implemented method of claim 1, comprising generating a therapy recommendation for the test subject based upon the prediction score output from the electronic neural network.

3. The computer-implemented method of claim 1, comprising administering a therapy to the test subject based upon the prediction score output from the electronic neural network.

4. The computer-implemented method of claim 1, wherein the oral cavity-related data comprises oral cavity images.

5. The computer-implemented method of claim 1, wherein the oral cavity-related data comprises image data, demographic data, symptom data, physical examination data, or a combination thereof.

6.-8. (canceled)

9. The computer-implemented method of claim 1, wherein the disease state comprises a bacterial infection, a viral infection, or a peritonsillar abscess.

10.-12. (canceled)

13. The computer-implemented method of claim 1, wherein the prediction score comprises a probability of a positive or negative streptococcus pharyngitis classification for the test subject.

14. The computer-implemented method of claim 1, wherein the oral cavity-related data comprises oral cavity images from the test and reference subjects, which oral cavity images comprise a region of interest selected from the group consisting of: a throat area, a tonsil area, a tongue area, a palate area, uvula area, posterior oropharynx area, lips area, cheek area, and neck area.

15.-20. (canceled)

21. The computer-implemented method of claim 1, wherein the oral cavity data from the test and reference subjects are obtained from videos of the test and reference subjects.

22.-25. (canceled)

26. The computer-implemented method of claim 1, wherein the first set of training data comprises oral cavity images and wherein the electronic neural network has been further trained on a second set of training data that comprises a plurality of sets of features extracted from numerical vectors representing sets of parameterized demographic data, symptom data, and/or physical examination data from the reference subjects and wherein the computer-implemented method further comprises passing a second set of features extracted from a numerical vector representing a set of parameterized demographic data, symptom data, and/or physical examination data from the test subject through the electronic neural network.

27. The computer-implemented method of claim 26, wherein the numerical vectors representing the set of parameterized demographic data, symptom data, and/or physical examination data from the reference subjects and from the test subject each comprise at least a 15-dimensional vector.

28. The computer-implemented method of claim 26, further comprising mapping the first and second sets of features to a bidimensional vector that corresponds to the prediction score for the disease state in the test subject.

29.-54. (canceled)

55. A system for generating a prediction score for a disease state in a test subject using an electronic neural network, the system comprising:
a processor; and
a memory communicatively coupled to the processor, the memory storing instructions which, when executed on the processor, perform operations comprising:
passing a first set of features extracted from oral cavity-related data obtained from a test subject through an electronic neural network, wherein the electronic neural network has been trained on a first set of training data that comprises a plurality of sets of features extracted from oral cavity-related data obtained from reference subjects, wherein the oral cavity-related data obtained from the reference subjects are each labeled with a positive or negative disease state ground truth classification for a given reference subject, and wherein one or more predictions for a positive or negative disease state classification for the given reference subject are made based on the oral cavity-related data obtained from the given reference subject, which predictions are compared to the ground truth classification for the given reference subject when the electronic neural network is trained; and
outputting from the electronic neural network a prediction score for the disease state in the test subject indicated by the first set of features extracted from the oral cavity-related data from the test subject.

56. The system of claim 55, wherein the instructions which, when executed on the processor, further perform operations comprising:
generating a therapy recommendation for the test subject based upon the prediction score output from the electronic neural network.

57. (canceled)

58. The system of claim 55, wherein the oral cavity-related data comprises image data, demographic data, symptom data, physical examination data, or a combination thereof.

59.-61. (canceled)

62. The system of claim 55, wherein the disease state comprises a bacterial infection, a viral infection, or a peritonsillar abscess.

63.-65. (canceled)

66. The system of claim 55, wherein the prediction score comprises a probability of a positive or negative streptococcus pharyngitis classification for the test subject.

67. The system of claim 55, wherein the oral cavity-related data comprises oral cavity images from the test and reference subjects, which oral cavity images comprise a region of interest selected from the group consisting of: a throat area, a tonsil area, a tongue area, a palate area, uvula area, posterior oropharynx area, lips area, cheek area, and neck area.

68.-75. (canceled)

76. The system of claim 55, wherein the first set of training data comprises oral cavity images and wherein the electronic neural network has been further trained on a second set of training data that comprises a plurality of sets of features extracted from numerical vectors representing sets of parameterized demographic data, symptom data, and/or physical examination data from the reference subjects and wherein the computer-implemented method further comprises passing a second set of features extracted from a numerical vector representing a set of parameterized demographic data, symptom data, and/or physical examination data from the test subject through the electronic neural network.

77.-100. (canceled)

101. A computer readable media comprising non-transitory computer executable instructions which, when executed by at least one electronic processor, perform at least:
passing a first set of features extracted from oral cavity-related data obtained from a test subject through an electronic neural network, wherein the electronic neural network has been trained on a first set of training data that comprises a plurality of sets of features extracted from oral cavity-related data obtained from reference subjects, wherein the oral cavity-related data obtained from the reference subjects are each labeled with a positive or negative disease state ground truth classification for a given reference subject, and wherein one or more predictions for a positive or negative disease state classification for the given reference subject are made based on the oral cavity-related data obtained from the given reference subject, which predictions are compared to the ground truth classification for the given reference subject when the electronic neural network is trained; and
outputting from the electronic neural network a prediction score for the disease state in the test subject indicated by the first set of features extracted from the oral cavity-related data from the test subject.

102. (canceled)
